(19) United States
(12) Patent Application Publication
(10) Pub. No.: US 2005/0107454 A1
(43) Pub. Date: May 19, 2005

(54) Process for the purification of climbazole

(75) Inventors: Alfred Korber, Holzminden (DE); Michael Michler, Eimen (DE); Heinz-Dieter Rheinlander, Lutgenade (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: Symrise GmbH & Co. KG.

(21) Appl. No.: 10/989,095

(22) Filed: Nov. 16, 2004

(30) Foreign Application Priority Data
Nov. 17, 2003 (DE) 103 53 673.6

(51) Int. Cl. C07D 233/54; A61K 31/4164

(52) U.S. Cl. 514/400; 548/341.5

(57) ABSTRACT

The invention relates to a process for the purification of climbazole and to climbazole-containing compositions produced in this way. Starting from a mixture containing climbazole and an organic solvent, climbazole is crystallised and the solvent separated off to obtain a crystal cake. The crystal cake is washed repeatedly with the organic solvent. During crystallising and washing, the crystal cake is freed from the solvent only to the extent that the crystal cake is still completely wetted with the solvent.

The present invention relates to a process for the purification of 1-(4-chlorophenoxy)-1-(1H-imidazolyl)-3,3-dimethyl-2-butanone (climbazole), and to compositions containing climbazole:

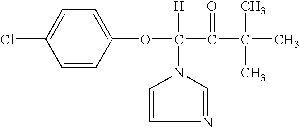

Climbazole is used in cosmetic preparations, and especially in shampoos, owing to its antimycotic action. Consequently, climbazole has to be present in the purest form possible; in particular, it must not be contaminated by accompanying substances which are not skin-compatible, impart an unpleasant odour or create an undesirable colour.

In DE 21 05 490 A, three processes for the production of imidazole derivatives are described, including in particular for climbazole. For the purification of the imidazole derivatives produced from a solution, it is taught, according to a process variant a), to evaporate off the solvent in vacuo and to take up the residue with a polar organic solvent. A water extraction should then take place to remove the by-product imidazolyl hydrochloride formed during the synthesis, and the solution should then be evaporated to dryness. The base is obtained from the residue by recrystallization and the salt by treating with the corresponding acid by conventional methods. As solvents in the production of the imidazole derivatives according to process a), polar organic solvents can preferably be used, especially nitriles such as azonitrile, sulfoxides such as dimethyl sulfoxide, formamides such as dimethylformamide, ketones such as acetone, ethers such as diethyl ether and tetrahydrofuran, nitroalkanes such as nitromethane, and unsymmetric chlorinated hydrocarbons such as methylene chloride and chloroform. In an example relating to the production of climbazole, it is described that the solvent is distilled off from a solution containing climbazole and the residue is washed three times with water. The remaining residue is then recrystallized from approximately 400 ml ligroin.

Regarding process variant b), it is taught to use a high-boiling organic solvent, particularly preferably aromatic hydrocarbons, such as e.g. xylene, or halogenated aromatic hydrocarbons, such as e.g. chlorobenzene. Using these solvents, water of reaction that forms can be separated off azeotropically. Process variant b) can also be carried out without solvents, e.g. in a melt. The purification of the desired imidazole derivatives takes place by conventional methods.

In a comparable manner, regarding process variant c) it is taught to use any inert organic solvents, preferably aromatic hydrocarbons, such as e.g. benzene, toluene, ethers, such as e.g. diethyl ether or tetrahydrofuran, chlorinated hydrocarbons, such as e.g. methylene chloride, chloroform and carbon tetrachloride, and lower alkyl nitriles, such as e.g. acetonitrile. Again, the purification of the desired imidazole derivatives takes place by conventional methods.

From EP 046 532, Example a), a process for the production of climbazole from 1-(4-chlorophenoxy)-3,3-dimethyl-1-fluoro-2-butanone with imidazole is known. According to the process described there under a), the two substances are heated to 130° C. for 3 hours. After cooling, the semi-solid residue is divided between methylene chloride/water, the organic phase is separated off and concentrated. The remaining crude end product is chromatographed through a small column with silicon dioxide.

In DE 29 37 595, the production and purification of climbazole are again described: 4-chlorophenol and sodium methylate are reacted in methanol with 1-bromo-3,3-dimethyl-1-(imidazol-1-yl)-butan-2-one hydrobromide. The residue is divided between ether and water, the organic phase is separated off, washed with sodium hydroxide solution, with water and with sodium chloride solution and dried over sodium sulfate. After distilling off the solvent, the residue is recrystallized from cyclohexane. The yield of climbazole is 70% of the theoretical value; melting point 95-97° C.

Because of the polar and nonpolar impurities resulting from the production processes, climbazole is conventionally recrystallized from isopropanol. In this case, climbazole is obtained in a yield of approx. 80% of the theoretical value in a purity of generally more than 98%.

Less polar solvents, such as e.g. toluene, give an approx. 10% better yield, but the end product is always contaminated by strong-smelling accompanying substances, consisting of pivalic acid among other substances.

In DE 197 03 651, the melt crystallization of climbazole and other imidazoles is described. The crystallization is performed here on one or more cooling surfaces, on which crystals of climbazole grow. The cooling process is terminated on reaching a desired crystal mass. The uncrystallized residual liquid, impoverished in climbazole, is removed from the cooling surfaces and the crystals that have formed. The crystals found on the cooling surfaces are melted on the surfaces by increasing the temperature. The resulting liquid, enriched with climbazole, is removed from the surfaces. The process is described explicitly using 2-methylimidazole as an example. Starting from a 2-methylimidazole grade with 99.1 wt. % purity and 9000 ppm impurities, crystallization is performed on a cooling surface in a temperature range of between 143 and 90° C. The crystals formed are melted off at a temperature of between 143 and 160° C. The melted crystals have a 2-methylimidazole content of 99.9 wt. % and an impurities content of 1000 ppm. It is a disadvantage of this process that the impurities cannot be separated off efficiently enough in the first melt crystallization and it is necessary to work in multiple steps.

In DE 196 18 578 C1, a process for the purification of climbazole is described in which crystallised climbazole in the form of a filter cake is washed with toluene that has been cooled to about 0° C. and the toluene is removed from the solid under pressure by supplying with nitrogen. An additional wash is then performed with approx. 5% sodium hydrogen carbonate solution. Finally, the filter cake is dried in vacuo in a drier by jacket heating. A yield of 90% is achieved.

It is a disadvantage of this process that, despite washing with aqueous sodium hydrogen carbonate, the crystalline climbazole still contains undesirable accompanying substances, for example 400-700 ppm 4-chlorophenol, and has a strong intrinsic odour, which is specific to the synthesis and mars the odour of cosmetic products. In particular, the accompanying substances often cause skin irritations or are undesirably allergenic. Furthermore, climbazole produced on an industrial scale often displays cloudiness after dissolving in organic solvents such as ethanol, which is caused by residual quantities of sodium hydrogen carbonate remaining in the product as a residue after drying the moist filter cake. The cloudiness is a problem when used in cosmetic products. In addition, climbazole produced in this way often possesses a Hazen colour index of 400-500 (greyish-white colour), which also causes problems with appearance when used in cosmetic products.

It was therefore an object of the present invention to provide a process for the processing or purifying of climbazole. In particular, frequently occurring accompanying substances from climbazole synthesis should be separated off to such an extent that they no longer cause problems with appearance when climbazole is used in cosmetic products. The accompanying substances from climbazole synthesis concerned are:

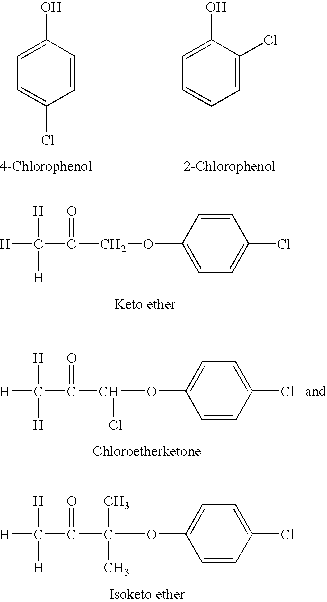

In particular, the content of 4-chlorophenol in the purified climbazole should be reduced to a level that is acceptable for cosmetic purposes. Furthermore, the purified climbazole should be substantially odourless, white, with a Hazen colour index <300 (measured in accordance with ISO 6271) and completely soluble in ethanol without any appreciable cloudiness.

According to the invention, therefore, a process for the purification of climbazole is provided, having the following steps:
a) starting from a mixture containing climbazole and a first organic solvent, crystallization of climbazole and separation of said first solvent to obtain a crystal cake, and
b) washing of the crystal cake thus obtained with a second organic solvent, the crystal cake being freed of the second solvent during crystallising and washing only to the extent that the crystal cake is still completely wetted with the second solvent. Step b) can be repeated one, two, or more times.

It is important for the result of the purification according to this process that the crystal cake remains substantially completely wetted with solvent during the washing, and thus remains moist. Thus, the crystal cake remains substantially completely wetted with the solvent both during the separation of the first organic solvent in step a) and during the separation of the second organic solvent during the washing in step b). Within the framework of the present invention, the crystal cake is considered to be completely wetted with a solvent even when an area of less than 10%, preferably less than 5% of the surface of the crystal cake that is externally visible is not wetted with the solvent; the 100% wetting of the crystal cake with the solvent is particularly advantageous. After the final washing at the end of the process, the second organic solvent is separated from the crystal cake to such an extent that this is no longer substantially completely wetted with the second organic solvent. For the washing in step b), the second organic solvent can be, and preferably is, the same as that used as the first organic solvent for the crystallizing in step a). It is also preferred to use the same solvent every time step b) is carried out.

It has been found that, after crystallizing out the climbazole by selective washing with the solvent in the manner just described, a climbazole-containing, solid composition can be obtained, which possesses the following properties:
Climbazole content: at least 98 wt. %, based on the total quantity of solids,
4-Chlorophenol content: no more than 400 ppm, preferably no more than 100 ppm, based on the total quantity of solids, and
Hazen colour index no more than 300, preferably no more than 200 (measured in accordance with ISO 6271).

The composition, therefore, consists essentially of pure climbazole. It is soluble to saturation in ethanol and forms a clear solution. In addition, compared with conventionally obtainable climbazole, it possesses a markedly reduced inherent odour, which is otherwise perceived as unpleasant. The composition obtainable according to the invention is therefore advantageously suitable for use in cosmetic preparations, especially creams and/or shampoos where conventionally prepared climbazole has previously been used or suggested. The climbazole prepared according to the present invention is suitable for the production of an antimycotic pharmaceutical preparation and/or antimycotic cosmetic preparation, especially a cream and/or a shampoo.

The process according to the invention therefore makes it possible for the first time to produce a composition of substantially pure climbazole, which has the advantageous properties just described. In addition, the process according to the invention can be carried out easily, requires no complicated melting processes and provides substantially pure climbazole in a high yield.

In step a) of the process, the temperature of the mixture to be crystallised can usefully be adjusted to −5 to 10° C., preferably to 0-3° C. At these temperatures, climbazole can be readily crystallized out of the organic solvents conventionally used in the synthesis. Alternatively or additionally, climbazole crystals can be added to inoculate the crystallization process.

Step b) of the process according to the invention is repeated at least once. In this way, the composition of substantially pure climbazole with the advantageous properties described above can be produced particularly easily and reliably. In general, it is sufficient for step b) to be performed twice more (total of three washings with the second organic solvent), while a particularly good purity of the composition is achieved if step b) is performed three additional times. In some cases, carrying out step b) up to four additional times may be advantageous.

For the purpose of washing, it is useful to use a solvent substantially free from accompanying substances which would be undesirable accompanying substances in the purified climbazole and, in particular, which can cause a problem if used in cosmetics by imparting an unpleasant odour, an undesirable colour or by causing skin irritations or triggering allergies. The solvent is therefore preferably free from the accompanying substances from climbazole synthesis listed above.

An organic solvent which is particularly suitable for use as the first or second organic solvent washing is selected independently from the group consisting of aromatic hydrocarbons, saturated hydrocarbons and polar organic solvents, or is a mixture of two or more members of this group. In particularly preferred embodiments, the organic solvent is selected from the group consisting of benzene, toluene, xylene, ethyl benzene, an alkane with up to 12 carbon atoms, methanol, ethanol, isopropanol, acetone, cyclohexanone, pentane, hexane, heptane, octane, isooctane, cyclohexane, methylcyclohexane and decalin or is a mixture of two or more members of this group. The water content in the solvent is preferably less than or equal to 5 wt. %, preferably less than or equal to 2 wt. %. The preferred solvent is toluene as the first and second organic solvent with at least industrial purity; this solvent makes a high climbazole yield possible with high purity of the climbazole obtained.

To carry out the process according to the invention, it is preferred if, in step b), the second organic solvent is applied to the crystal cake for the purpose of washing in a ratio by volume of solvent to crystal cake of 0.5:1 to 5:1, preferably 0.8:1 to 1.5:1. This application preferably takes place by covering the crystal cake with a layer of the second organic solvent. The crystal cake is preferably covered with a quantity of solvent equal to or up to twice the layer thickness of the crystal cake. If the quantities of solvent described are maintained for washing the crystal cake, particularly pure climbazole-containing compositions are obtained in a high yield.

It is also preferred to smooth the crystal cake after separating off the solvent in step a) and/or b). This can usefully take place with the blades of an agitator. By smoothing, a crystal cake of uniform thickness is produced. The crystal cake treated in this way makes it easier to remove the solvent in a subsequent washing step in such a way that the crystal cake as a whole is uniformly wetted with the solvent. In addition, as a result of the smoothing, an area of the crystal cake of no more than 10% of the externally visible surface of the crystal cake, which is no longer wetted with the solvent after separating off the solvent, can be brought into contact with wetted areas of the crystal cake and wetted with solvent again in this way so that the crystal cake is, as a result, substantially uniformly wetted with solvent.

Accordingly, it is also preferred to stir the climbazole-containing mixture during the separating of the first organic solvent in step a). In this way, the formation of a uniformly thick crystal cake is advantageously supported.

Those embodiments of the process according to the invention in which the crystal cake is distributed on a filter to perform step b) and the solvent is removed under pressure in step a) and/or in step b) by applying gas pressure, without gas breakthrough, are particularly preferred. In this embodiment, the crystal cake forms a filter cake on the filter. In this way, the solvent can be removed particularly easily. In addition, by adjusting the gas pressure and the duration of the gas supply, it is particularly easy to ensure that the crystal cake is still completely wetted with the solvent after separating off the solvent. As gases for applying the gas pressure, inert gases are preferably used, such as e.g. compressed air, argon, nitrogen, with nitrogen being particularly preferred. The excess pressure of the gas while applying the gas pressure is advantageously 0.5 to 2 bar, preferably 0.8 to 1.2 bar, particularly preferably 1 bar.

The temperature of the second organic solvent is adjusted at the beginning of step b) to −5 to 5° C., preferably 0-3° C., in preferred embodiments of the process according to the invention. With solvents at these temperatures, high yields and a good purity of the climbazole-containing composition can be achieved.

Unless anything to the contrary has been expressly stated above, the embodiments of the process according to the invention described can be combined with one another at will. In particular, to purify climbazole, a process with the following steps is preferred:
starting from a mixture containing climbazole and a first organic solvent, preferably toluene and/or n-heptane, crystallizing of climbazole,
distributing the mixture on a filter and separating the solvent by applying gas pressure with 0.8 to 1.2 bar nitrogen, without gas breakthrough, to obtain a crystal cake, and
washing the crystal cake thus obtained twice with a second organic solvent, preferably toluene and/or n-heptane, the crystal cake being covered with a layer of second organic solvent equal to or up to twice the layer thickness of the crystal cake, and the solvent being separated off by applying gas pressure with 0.8 to 1.2 bar nitrogen, without gas breakthrough.

The invention is described below in more detail on the basis of the examples:

### EXAMPLE 1

### Crystallization and Purification of Climbazole in Toluene

Toluene in an amount of 2,500.0 kg is initially charged into a 5 m³ dissolving tank, and 3,000 kg of 92% climbazole crude are added via a solids filler. The suspension is heated to 80-85° C., whereupon the crude climbazole dissolves. The solution is circulated through a filter to remove traces of insoluble solids. The clear filtrate is pumped into a 5 m³ crystallizing tank.

In the crystallizing tank, the solution is adjusted to 0-3° C. by inoculating with climbazole crystals and slowly cooling. The crystal suspension is pumped into a filter dryer that has been cooled to 0° C. with stirring. The agitator is adjusted to such a low stirring speed that a filter cake of uniform thickness can form.

The liquid phase above the filter cake is removed under pressure, using 1 bar nitrogen excess pressure, only to the extent that no gas breakthrough takes place and the remaining crystal cake remains wetted with solvent. The excess pressure is then released. The filter cake is smoothed with the agitator blades to close any cracks that may have formed in the filter cake. The agitator is then switched off.

The crystal cake is covered with a layer of 1,000 litres of cold toluene at 0-3° C. The toluene layer is again removed under pressure, using 1 bar excess nitrogen pressure, without a gas breakthrough occurring.

The last operation is repeated 2 more times with 600 litres toluene, pressure being applied during the final rinsing of the filter cake until gas breakthrough takes place and 30 minutes beyond.

The filter cake is then dried in the same piece of apparatus while stirring, applying a vacuum and heating.

Yield: 2,580.0 kg=93.5% of theoretical value

Purity of the climbazole: 99.1%

White crystals are obtained, which are free from chlorophenols and odourless. The crystals form a clear solution in ethanol with no residue.

### EXAMPLE 2

### Crystallization and Purification of Climbazole in N-Heptane

N-heptane in an amount of 3,000.0 kg is initially charged into a 5 m³ dissolving tank and 1,500.0 kg of 92% climbazole crude are added via a solids filler. The suspension is heated to 80-85° C., whereupon the crude climbazole dissolves. The solution is circulated through a filter to remove traces of insoluble solids. The clear filtrate is pumped into a 5 m³ crystallising tank.

In the crystallizing tank, the solution is adjusted to 0-3° C. by inoculating with climbazole crystals and slowly cooling. The crystal suspension is washed as in Example 1 with 700 litres and twice with 400 litres of n-heptane by displacement washing.

Yield: 1,290.0 kg=92.4% of theoretical value

Purity of the climbazole: 99.0%

White crystals are obtained, which are free from chlorophenols and odourless. The crystals form a clear solution in ethanol with no residue.

### EXAMPLE 3

### Crystallization and Purification of Climbazole in Isopropanol

Isopropanol in an amount of 200 g and 300 g of 92% climbazole crude are added to a 500 ml glass flask. The flask is heated to reflux boiling point. The solution is slowly cooled in an ice bath to 0-3° C. The precipitated crystals are separated from the solvent by means of a suction filter.

The filter cake is washed 2 more times with cold isopropanol at 0° C. in the manner described in Examples 1 and 2, the first displacement wash being performed with 100 ml isopropanol and the second with 60 ml isopropanol.

Yield: 240 g=87.3% of theoretical value

Purity of the climbazole: 99.3%

White crystals are obtained, which are free from chlorophenols and odourless. The crystals form a clear solution in ethanol with no residue.

### EXAMPLE 4

### Overview of Various Climbazole Compositions Produced According to the Invention

| | | 2-Chloro- | 4-Chloro- | 2,4-Dichloro- | 3,4-Dichloro- | | |
|---|---|---|---|---|---|---|---|
| | Climbazole | phenol | phenol | phenol | phenol | Hazen | |
| | content | content | content | content | content | colour | |
| Sample | [%] | [%] | [%] | [%] | [%] | index | Odour |
| 1 | 99.5 | 0 | 0.003 | 0 | 0 | 158 | A-B |
| 2 | 98.7 | 0 | 0.002 | 0 | 0 | 193 | A-B |
| 3 | 99 | 0 | 0.002 | 0 | 0 | 198 | A-B |
| 4 | 98.1 | 0 | 0.004 | 0 | 0 | 202 | A-B |
| 5 | 98.1 | 0 | <0.001 | 0 | 0 | 178 | A-B |

Odour evaluation: A: Odour good; B: Odour acceptable; C: Odour unacceptable

### COMPARATIVE EXAMPLE

### Overview of Conventional Climbazole Compositions

| | | 2-Chloro- | 4-Chloro- | 2,4-Dichloro- | 3,4-Dichloro- | | |
|---|---|---|---|---|---|---|---|
| | Climbazole | phenol | phenol | phenol | phenol | Hazen | |
| | content | content | content | content | content | colour | |
| Sample | [%] | [%] | [%] | [%] | [%] | index | Odour |
| 1 | 99.4 | 0 | 0.045 | 0 | 0 | 504 | C |
| 2 | 99.1 | 0 | 0.063 | 0 | 0 | 442 | C |

1. Process for the purification of climbazole, comprising the steps:
a) starting from a mixture containing climbazole and a first organic solvent, crystallizing climbazole and separating said first organic solvent to obtain a crystal cake, and
b) washing the crystal cake thus obtained with a second organic solvent, characterized in that
the crystal cake is freed of the solvent during crystallising and washing only to the extent that the crystal cake is still completely wetted with the solvent, and that
step b) is repeated.

2. Process according to claim 1, wherein the second organic solvent used for the washing is selected from the group consisting of aromatic hydrocarbons, saturated hydrocarbons and polar organic solvents, or a mixture of two or more members of this group.

3. Process according to claim 2, wherein the second organic solvent used for the washing is selected from the group consisting of benzene, toluene, xylene, ethylbenzene, an alkane with up to 12 carbon atoms, methanol, ethanol, isopropanol, acetone, cyclohexanone, pentane, hexane, heptane, octane, isooctane, cyclohexane, methylcyclohexane and decalin or is a mixture of two or more members of this group.

4. Process according to claim 1, wherein the second organic solvent is applied to the crystal cake for the purpose of washing in step b) in a ratio by volume of solvent to crystal cake within the range of 0.5:1 to 5:1.

5. Process according to claim 1, wherein the crystal cake is smoothed after separating off the solvent.

6. Process according to claim 1, characterized by stirring while separating off the first organic solvent in step a).

7. Process according to claim 1, wherein the crystal cake is distributed on a filter to perform step b) and the solvent is removed under pressure in step a) and/or in step b) by applying gas pressure, without gas breakthrough.

8. Process according to claim 1, wherein the temperature of the organic solvent is adjusted at the beginning of step b) to a temperature within the range of −5 to 5° C.

9. A climbazole-containing composition produced by a process according to claim 1.

10. A climbazole-containing composition with the following properties:
climbazole content: at least 98 wt. %, based on the total quantity of solids,
4-chlorophenol content: no more than 400 ppm, based on the total quantity of solids, and
Hazen colour index no more than 300.

11. A cosmetic composition comprising a climbazole-containing composition according to claim 10.

12. A pharmaceutically acceptable composition comprising a climbazole-containing composition according to claim 10.

13. Process according to claim 4, characterized in that the second organic solvent is applied to the crystal cake for the purpose of washing in step b) in a ratio by volume of solvent to crystal cake within the range of 0.8:1 to 1.5:1.

14. Process according to claim 8, wherein the temperature of the organic solvent is adjusted at the beginning of step b) to a temperature within the range of 0-3° C.
